(19) United States
(12) Patent Application Publication
(10) Pub. No.: US 2008/0161773 A1
(43) Pub. Date: Jul. 3, 2008

(54) Apparatus and method for delivering ophthalmic fluid

(76) Inventors: Nancy Melberg Holekamp, St. Louis, MO (US); David Cy Beebe, St. Louis, MO (US); Ying-Bo Shui, St. Louis, MO (US); Steven Bassnett, Chesterfield, MO (US); Richard McNulty, Kingsford (AU)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 12/009,434

(22) Filed: Jan. 18, 2008

Related U.S. Application Data
(63) Continuation-in-part of application No. 10/836,569, filed on May 1, 2004.

(51) Int. Cl. A61F 9/00; A61M 5/14

(52) U.S. Cl. 604/521; 604/257

(57) ABSTRACT

An apparatus for delivering infusion fluid into an eye includes a container defining a chamber configured to contain a quantity of infusion fluid. A first tube defines a first passage that provides fluid communication between the chamber and a volume defined within the eye. At least a portion of the quantity of infusion fluid is infused into the volume through the first passage. A second tube is positioned about the first tube and defines a second passage between an outer surface of the first tube and an inner surface of the second tube. A quantity of at least one substantially oxygen-free inert gas is contained within the second tube to facilitate maintaining the infusion fluid in a low oxygen state. In an alternative embodiment, a vacuum is produced within the second tube to facilitate maintaining the infusion fluid in a low oxygen state. In a further alternative embodiment, the apparatus includes only one tube that is substantially impermeable to oxygen to facilitate maintaining the infusion fluid in a low oxygen state.

### CROSS REFERENCE TO RELATED APPLICATIONS

This application is a continuation-in-part of U.S. application Ser. No. 10/836,569, filed May 1, 2004, which is hereby incorporated by reference in its entirety.

### STATEMENT REGARDING FEDERALLY SPONSORED RESEARCH & DEVELOPMENT

This research was partially funded by US NIH grant EY04853 and EY015863. The U.S. Government may have certain rights in the invention.

### BACKGROUND OF THE INVENTION

This invention relates generally to medical procedures involving the eye and, more particularly, to an apparatus and method for delivering infusion fluids, such as an ophthalmic fluid, to an eye during eye surgery.

The vitreous is a transparent gel that fills at least a portion of the eye, typically from the iris to the retina. The vitreous helps the eye hold its shape. The vitreous is susceptible to several afflictions that impair vision by damaging its transparency. Some of these afflictions are infections, injuries, bleeding, and blood vessels growing into the vitreous. In addition, on occasion the retina will fall into the vitreous, a condition called retinal detachment. For any of these above listed afflictions and for other afflictions of the retina that require retinal surgery, it may be necessary to surgically remove the vitreous and substitute a suitable solution during the surgery. The removal and substitution procedure is called vitrectomy. The solution is added to the eye at a sufficient rate such that the eye maintains its pressure and shape during and after the procedure.

During a typical vitrectomy procedure three probes (a vitrector, a light source, and an infusion cannula) are inserted into the eye through small incisions in the sclera, a tough outer coating of the eye. The vitreous gel and fluid is removed using the vitrector, a miniature cutting and aspiration tool. The infusion line serves to keep the pressure constant in the eye during the procedure. As the vitreous is aspirated, an infusion fluid is provided to the eye with a slight positive pressure through the infusion cannula. During the operation (which may take several hours), a total of approximately 100 ml to approximately 400 ml of solution is infused into the eye and aspirated along with the vitreous. The infusion fluid is typically a physiologic saline solution similar in properties to the material being removed from the eye. A notable exception is that the solution is oxygenated (partially equilibrated with room air) and is supplied to the eye at a temperature commensurate with the room temperature of the operating room, i.e., around 24° C. to about 27° C.

Although vitrectomy is an effective treatment, it is associated with an extremely high incidence of post-surgical cataracts. For example, within six months of vitrectomy, approximately 21% of patients develop cataract and within 12 months of the vitrectomy, this number rises to approximately 63%. The most common type of cataract seen after vitrectomy is nuclear cataract. Eventually, cataract surgery is required in a majority of the cases.

### BRIEF DESCRIPTION OF THE INVENTION

In one aspect, an apparatus for delivering infusion fluid into an eye is provided. The apparatus includes a container defining a chamber configured to contain a quantity of infusion fluid. A first tube defines a first passage providing fluid communication between the chamber and a volume defined within the eye. At least a portion of the quantity of infusion fluid is infused into the volume through the first passage. A second tube is positioned about the first tube and defines a second passage between an outer surface of the first tube and an inner surface of the second tube. A quantity of at least one substantially oxygen-free inert gas is contained within the second tube to facilitate maintaining the infusion fluid in a low oxygen state.

In another aspect, a method for delivering an infusion fluid from a container into a volume defined within an eye is provided. The method includes removing oxygen from within the infusion fluid as the infusion fluid flows from a chamber defined by the container into the volume through a first tube providing fluid communication between the chamber and the volume.

In another aspect, a method for delivering ophthalmic fluid into an eye is provided. The method includes providing a container defining a chamber configured to contain deoxygenated ophthalmic fluid. A quantity of ophthalmic fluid is infused from the container into the eye through a first tube defining a first passage providing fluid communication between the chamber and a volume defined within the eye. A second tube is positioned about the first tube. The second tube defines a second passage between an outer surface of the first tube and an inner surface of the second tube. The second tube contains at least one substantially oxygen-free inert gas to facilitate maintaining the ophthalmic fluid in a low oxygen state.

In another aspect, a method for infusing a deoxygenated fluid from a container into a volume defined within an eye is provided. The method includes infusing at least a portion of the deoxygenated fluid into the volume through a passage defined by a first tube providing fluid communication between a chamber defined by the container and the volume. A second tube is positioned about the first tube. The second tube defines a second passage between an outer surface of the first tube and an inner surface of the second tube. A vacuum is produced within the second tube to facilitate maintaining the deoxygenated fluid in a low oxygen state.

In another aspect, a method for delivering a deoxygenated fluid from a container into a volume defined within an eye is provided. The method includes infusing at least a portion of the deoxygenated fluid into the volume through a passage defined by a tube providing fluid communication between a chamber defined by the container and the volume. The tube is made of a substantially oxygen-impermeable material.

### BRIEF DESCRIPTION OF THE DRAWINGS

FIG. 1 is a set of graphs depicting the effect of temperature on PO₂ in the nucleus of a human donor lens; and

FIG. 2 is a schematic partial sectional view of an exemplary apparatus for delivering ophthalmic fluid into a volume defined by an eye; and

FIG. 3 is a schematic view of an alternative exemplary apparatus for delivering ophthalmic fluid into a volume defined by an eye.

### DETAILED DESCRIPTION OF THE INVENTION

The present invention provides an apparatus and method for delivering infusion fluid, such as ophthalmic fluid, into a volume defined within an eye. The apparatus and method of the present invention prevents or limits the introduction of oxygen into the infusion fluid, as the infusion fluid is transferred from an infusion fluid bottle or container into the eye volume. Further, in one embodiment, oxygen is removed from within the infusion fluid as the infusion fluid flows from a chamber defined by the container into the volume through a first tube providing fluid communication between the chamber and the volume.

Exemplary embodiments of compositions of fluids and methods of supplying the fluids are described below. In one embodiment, the fluid includes reduced glutathione and the method includes deoxygenating the fluid and adding reduced glutathione to the fluid. In another embodiment, the fluid includes ascorbate in quantities as high as 2 mM. The fluid is supplied to the eye through an oxygen impermeable tube within a temperature range of about 30° C. to about 37° C. to the eye. Although exemplary embodiments are described herein, the fluid and methods of delivery are not limited to those specific embodiments.

A factor causing damage to an eye during surgery is the traumatic change in environment. Exposure to the atmosphere or to a foreign wetting solution presents a possibly hostile environment for the cells in the eye. For example, exposure to a particular fluid may present a challenge for the cells involved. Accordingly, solutions are provided to the eye during a vitrectomy that attempt to approximate the natural bodily fluids in the eye.

It is believed that fluid delivered to the eye at conditions that mimic the conditions in the eye, will reduce the occurrence of post-vitrectomy nuclear cataracts. Such conditions include a temperature at or near body temperature and an oxygen level at or near the oxygen level of the vitreous body gel that is being replaced. The fluid may also include ascorbate concentrations approximating the ascorbate concentrations of the vitreous gel, about 2 mM.

Due to experiments recently conducted on cadaveric lenses, it is believed that the lens core (the center tissue in the lens) is flooded with oxygen during a typical vitrectomy, due, at least in part, to the cooling of the lens in the presence of oxygenated solutions. It is believed that the introduction of high concentrations of oxygen into the previously hypoxic core of the lens is a direct cause of accelerated growth of post-operative cataracts. Two aspects of the fluid are believed to play a role in providing the high concentration of oxygen to the lens. The first aspect is that fluid is typically provided to the lens in an oxygenated state (partially equilibrated with room air). The second aspect is that the fluid is delivered to the eye at room temperature (typically between about 24° C. and 27° C.). The cool temperature of the fluid causes the lens to cool which allows more oxygen to enter the lens.

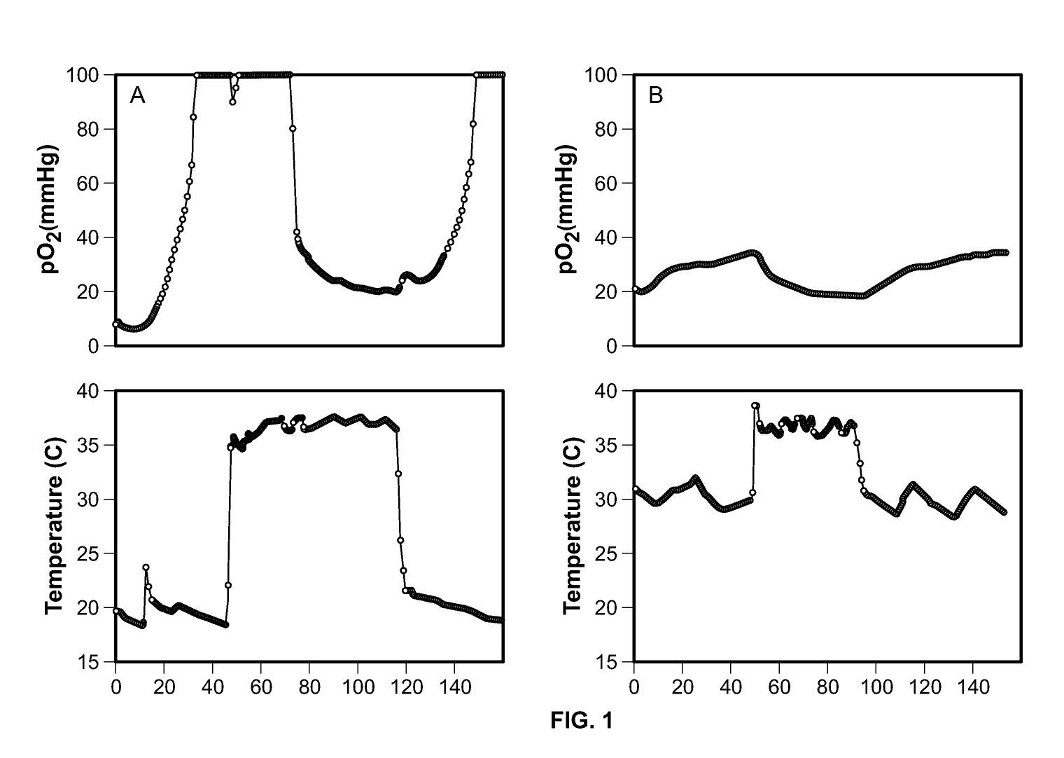

FIG. 1 illustrates the effect of temperature on oxygen content (pO₂) in the nucleus of human donor lenses. Isolated human lenses were incubated in solutions equilibrated with atmospheric oxygen (about 150 mmHg or 21% O₂)—In A, the pO₂ and temperature were monitored in the lens center as the temperature of the bathing solution was alternated between 37° C. and 20° C. At 37° C., pO₂ in the lens core is less than 10 mmHg. At room temperature, the pO₂ at the lens core increases to greater than 100 mmHg. The effect appears to be reversible. In B, the lens core is maintained in a relatively hypoxic condition (less than 35 mmHg) provided the tissue temperature does not fall below 30° C.

In accordance with one embodiment of the present invention, an ophthalmic fluid is prepared for performing a surgery on an eye. The ophthalmic fluid is deoxygenated and then stored in an oxygen impermeable storage bottle. The process of deoxygenating the fluid includes introducing nitrogen gas into the fluid so that the nitrogen replaces most of the oxygen. The nitrogen gas is delivered from a tank of compressed pure nitrogen and is passed through a 0.45 micrometer filter to prevent contaminants from entering the solution. The nitrogen gas enters the bottle containing the fluid through a port in the bottle. The nitrogen gas is bubbled through the fluid and exits through an opening at the top of the bottle. Nitrogen is bubbled for approximately 10 minutes to remove the majority of the oxygen. In one embodiment, nitrogen is bubbled through the fluid until the oxygen partial pressure is less than about 10 mmHg. In another embodiment, nitrogen is bubbled through the fluid until the oxygen partial pressure is between about 10 mmHg and about 2 mmHg. More particularly, nitrogen is bubbled through the fluid until the oxygen partial pressure is about 5 mmHg or less (approximately 0.5% oxygen). In another embodiment, nitrogen is bubbled through the fluid until the oxygen partial pressure is less than about 2 mmHg.

In an exemplary embodiment, 500 cc of sterile ophthalmic fluid is contained in an oxygen impermeable bottle, e.g., glass, which is capped with an oxygen impermeable rubber stopper. A sterile needle of approximately 18 gauge bore and 6 inches long is used to penetrate the rubber stopper so the needle rests within the fluid. The end of the needle outside the bottle is attached to tubing connected to a tank of 100% nitrogen gas. Under low pressure nitrogen gas is bubbled into the ophthalmic fluid for at least 10 minutes. Also inserted through the rubber stopper alongside the needle is a one-way valve for the egress of excess oxygen or nitrogen gases during the bubbling procedure. The bubbling of nitrogen gas in the ophthalmic fluid as described above will result in a fluid oxygen tension of 2 to 10 mm Hg pressure, thus effectively deoxygenating the fluid.

In one embodiment, the fluid is deoxygenated in the same location at which it is formed. For example, after the solution has been created, it is immediately deoxygenated and then stored in an oxygen impermeable storage bottle. Alternatively, the fluid is deoxygenated at the location at which the procedure is to be performed.

A first exemplary ophthalmic fluid is set forth below in Table 1.

TABLE 1

| Component | Amount (Wt %) |
|---|---|
| sodium chloride | 0.64% |
| potassium chloride | 0.075% |
| calcium chloride dihydrate | 0.048% |
| magnesium chloride | 0.03% |
| sodium acetate | 0.39% |
| sodium citrate dihydrate | 0.17% |
| sodium hydroxide | to adjust pH in water |
| or hydrochloric acid | |

A fluid having the composition as listed in Table 1 is currently available from Alcon Laboratories, Inc., Fort Worth, Tex. 76134. A second exemplary ophthalmic fluid is set forth below in Table 2.

TABLE 2

| Component | Amount (mg/ml) |
|---|---|
| sodium chloride | 07.14 mg |
| potassium chloride | 0.38 mg |
| calcium chloride dihydrate | 0.154 mg |
| magnesium chloride | 0.20 mg |
| dibasic sodium phosphate | 0.42 mg |
| sodium bicarbonate | 2.10 mg |
| dextrose | 0.92 mg |
| glutathione | 0.184 mg |
| sodium hydroxide | to adjust pH in water |
| or hydrochloric acid | |

A fluid having the composition as listed in Table 2 is currently available from Alcon Laboratories, Inc., Fort Worth, Tex. 76134.

In one embodiment, after the ophthalmic fluid has been deoxygenated, at least one reducing agent is added to the fluid. An exemplary reducing agent is reduced glutathione. The glutathione is added to the fluid by injecting the glutathione into the storage bottle containing the deoxygenated fluid in an amount that provides the desired final concentration of reduced glutathione. Alternatively, the reduced glutathione is combined with the deoxygenated fluid during infusion with a proportioning pump that adds an appropriate amount of reduced glutathione to the fluid during infusion. In a further embodiment, the glutathione is added to the fluid by other means that result in the desired final concentration of glutathione in the fluid entering the eye. For example, 10 mM sterile, reduced glutathione is dissolved in the infusion fluid prior to infusing the fluid into the eye.

Although reduced glutathione has been described as a reducing agent in the exemplary embodiment, it should be understood that other reducing agents can also be used. Other exemplary reducing agents that can be used in the present invention include: ascorbic acid (vitamin C), N-acetyl cysteine, D- or L-cysteine, D- or L-methionine, dithiothreitol, dithioerythritol, and mercaptoethanol.

The method for providing deoxygenated fluid to the eye includes deoxygenating the fluid as described above. A reducing agent is then added to the deoxygenated fluid. The fluid is then delivered to the eye through an oxygen-impermeable tubing during the procedure under a positive pressure. The positive pressure is controlled, in one embodiment, with a pneumatic pump within the vitrectomy machine. The gas chosen to power the pneumatic pump is nitrogen since the gas from the pump may contact the fluid.

Another aspect of the invention is that the fluid is warmed prior to infusion into the eye. The temperature of the warmed fluid as it enters the eye is between about 30° C. and about 37° C. More particularly, the temperature of the warmed fluid as it enters the eye is between about 32° C. and about 35° C. More particularly still, the temperature of the warmed fluid as it enters the eye is about 34° C.

In an exemplary embodiment, the fluid is warmed by a fluid warming device. An exemplary device is WarmFlo, model FW-538 and WF-100 (Mallinckrodt, Inc., St. Louis, Mo.). The fluid warmer utilizes a sterile, disposable, heat exchange cassette. Similar devices are used to warm blood and I.V. solutions to help maintain normal body temperature of patients undergoing surgical procedures. In use, the infusion fluid is passed through the fluid warmer, which is positioned relatively close to the patient's head. The positioning helps to alleviate cooling of the fluid after it has been warmed. In one embodiment, the fluid warmer is equipped with an audible over-temperature alarm. If the alarm sounds, then the fluid flow to the eye is quickly turned off.

The method of providing warmed infusion fluid to the eye includes attaching the fluid warmer to a drip stand and placing the warmer near the head of the patient. The temperature for the warmed fluid is set to between about 30° C. and about 37° C. In one aspect, the temperature for the warmed fluid is set to between about 32° C. and about 35° C. In a further aspect, the temperature for the warmed fluid is set to about 34° C. The irrigation solution bottle is attached to the sterile irrigation tubing as in a normal vitrectomy. The irrigation tubing is attached to the proximal end of the sterile heat exchange unit. The distal end of the heat exchange unit is attached to a standard 3-way tap and silicon tubing going to the eye. The irrigation tubing and heat exchange unit is primed with irrigation solution. The vitrectomy is then conducted. This warming method can be practiced with a deoxygenated fluid by replacing the silicon tubing with an oxygen impermeable tubing, thus keeping oxygen out of the fluid during its transit from the storage bottle to the eye.

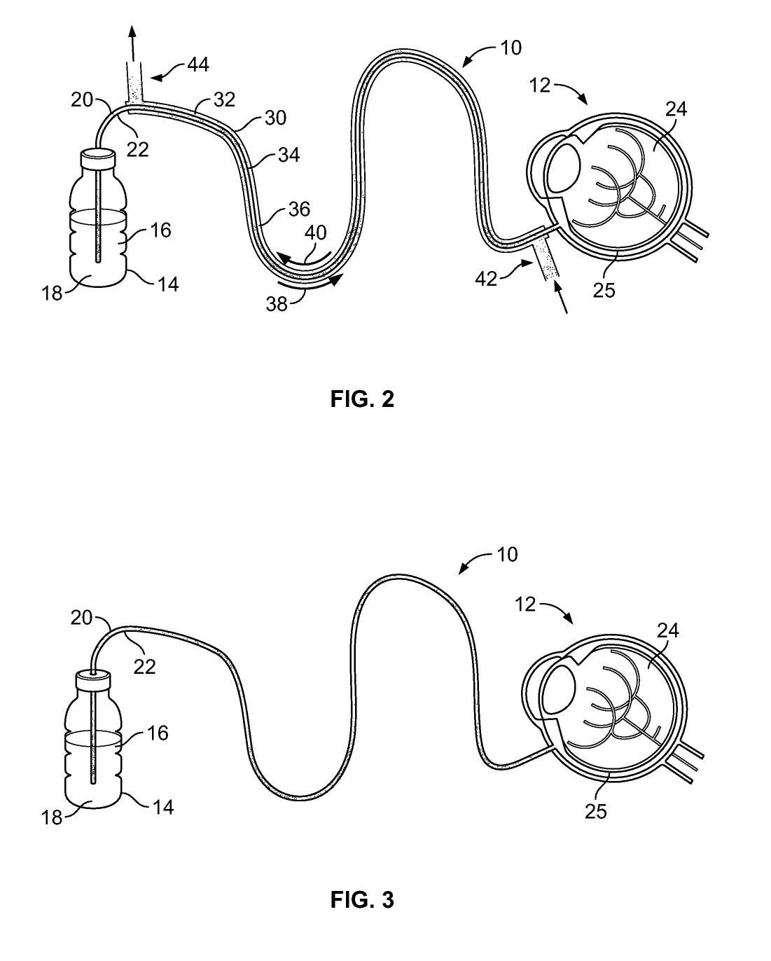

FIG. 2 shows an apparatus 10 for delivering infusion fluid into an eye 12, in accordance with one embodiment of this invention. Apparatus 10 includes a container 14 that defines a chamber 16 configured to contain a quantity of infusion fluid 18, such as a deoxygenated ophthalmic fluid. In one embodiment, infusion fluid 18 includes at least one supplement, such as reduced glutathione and/or ascorbic acid. In a particular embodiment, container 14 is an oxygen impermeable storage bottle.

Apparatus 10 includes an inner tube 20 having a first end that extends into chamber 16. Inner tube 20 is made of a suitable irrigation tubing. It should be apparent to those skilled in the art and guided by the teachings herein provided that inner tube 20 may extend into chamber 16 through a suitable port or opening in container 14. Alternatively, inner tube 20 is coupled to a suitable extension tube, which extends into chamber 16 to draw infusion fluid, as desired. Inner tube 20 defines a first passage 22 that provides fluid communication between chamber 16 and a volume 24 defined within eye 12. Referring to FIG. 2, eye 12 defines or forms volume 24 within which the vitreous 25 is contained to form and/or shape eye 12. During a typical virectomy procedure, vitreous 25 is aspirated and a quantity of infusion fluid 18 is infused into volume 24 through first passage 22.

As shown in FIG. 2, an outer tube 30 is positioned about inner tube 20. Outer tube 30 defines a second passage 32 between an outer surface 34 of inner tube 20 and an inner surface 36 of outer tube 30. In one embodiment, a suitable substantially oxygen-free inert gas, such as nitrogen gas, a noble gas or a mixture thereof, is contained within second passage 32 to prevent or limit introduction or diffusion of oxygen through the wall of inner tube 20 and into deoxygenated infusion fluid 18 within first passage 22 to maintain deoxygenated infusion fluid 18 in a low oxygen state. As used herein, the phrase “low oxygen state” refers to an oxygen partial pressure that is at least one of less than about 10 mmHg, between about 10 mmHg and about 2 mmHg, about 5 mmHg or less (approximately 0.5% oxygen), and less than about 2 mmHg.

In a further embodiment, the substantially oxygen-free inert gas is urged to move or flow through second passage 32 to facilitate removing oxygen from infusion fluid 18 moving or flowing through first passage 22. A stream of the substantially oxygen-free inert gas is directed to flow through second passage 32. The flow of the substantially oxygen-free inert gas through outer tube 30 facilitates diffusion of oxygen within infusion fluid 18, flowing through first passage 22, through the wall of inner tube 20 and into the stream of substantially oxygen-free inert gas flowing through second passage 32. The substantially oxygen-free inert gas may include any suitable gas or mixture of suitable gases, such as nitrogen, a noble gas or combinations thereof. In a particular embodiment, infusion fluid 18 flows in a first direction through inner tube 20, as shown by arrow 38 in FIG. 2, and the substantially oxygen-free inert gas flows through outer tube 30 in a second direction, as shown by arrow 40, substantially opposite the first direction to actively deoxygenate infusion fluid 18 within inner tube 20 that includes oxygen.

In a particular embodiment, the substantially oxygen-free inert gas consists essentially of nitrogen gas. The nitrogen gas is directed through outer tube 30 for facilitating diffusion of oxygen within infusion fluid 18 through inner tube 20 and into the stream of nitrogen gas. The oxygen concentration of the nitrogen gas is substantially zero and, thus, provides a diffusion gradient across inner tube 20 by which oxygen within infusion fluid 18 flowing through inner tube 20 diffuses across inner tube 20 into second passage 32. In this embodiment, the nitrogen gas enters second passage 32 at a distal end 42 of outer tube 30, e.g., at an end of outer tube 30 generally opposite container 14 containing infusion fluid 18. The nitrogen gas exits to the atmosphere at a proximal end 44 of outer tube 30 at or near the coupling of inner tube 20 to container 14 to maximize an efficiency of oxygen removal from infusion fluid 18. The emitted gas includes oxygen removed from within infusion fluid 18 as a result of diffusion. This “countercurrent flow” facilitates efficient removal of oxygen present in infusion fluid 18 as infusion fluid 18 flows through inner tube 20. Because infusion fluid flow through inner tube 20 is relatively slow during a surgical procedure, in one embodiment, an oxygen level within infusion fluid 18 is reduced from about 55 mmHg (the oxygen level of infusion fluid 18 as commercially provided for clinical use) to about 0 mmHg.

In an alternative embodiment, outer tube 30 does not contain an inert gas but, rather, a vacuum is produced within outer tube 30 to maintain deoxygenated infusion fluid 18 contained within inner tube 20 in a low oxygen state. It should be apparent to those skilled in the art and guided by the teachings herein provided that any suitable vacuum pump or mechanism may be utilized to produce a suitable vacuum within outer tube 30.

Referring to FIG. 3, in a further alternative embodiment, apparatus 10 includes only one tube, such as inner tube 20 defining passage 22, having a first end that extends into chamber 16. Inner tube 20 is made of a suitable single lumen tube that is substantially impermeable to oxygen. In a particular embodiment, inner tube 20 is made of a flexible metal tubing material having a composition that serves as a barrier to oxygen. Alternatively, inner tube 20 may be made of a suitable oxygen-impermeable plastic or polymer-based tubing material. It should be apparent to those skilled in the art and guided by the teachings herein provided that inner tube 20 may include any suitable material that is substantially impermeable to oxygen. In one embodiment, inner tube 20 extends into chamber 16 through a suitable port or opening in container 14. Alternatively, inner tube 20 is coupled to a suitable extension tube, which extends into chamber 16 to draw deoxygenated infusion fluid 18, as desired. Passage 22 provides fluid communication between chamber 16 and volume 24 defined within eye 12. Because inner tube 20 is made of a material that is substantially impermeable to oxygen, deoxygenated infusion fluid 18 remains substantially deoxygenated as infusion fluid 18 moves through inner tube 20 into volume 24.

In an exemplary embodiment, infusion fluid 18 is warmed to a temperature between about 30° C. and about 37° C. In one aspect, the temperature for the warmed infusion fluid 18 is set to between about 32° C. and about 35° C. In a further aspect, the temperature for the warmed infusion fluid 18 is set to about 34° C. Infusion fluid 18 is warmed as infusion fluid 18 passes through first passage 22 of inner tube 20. Using a suitable method, inner tube 20 itself is warmed and/or the inert gas passing through second passage 32, e.g., nitrogen gas, or the vacuum produced within second passage 32 is warmed. Alternatively, infusion fluid 18 is warmed within chamber 16 and then passed trough an insulated tubing to maintain an infusion fluid temperature of about 34° C. as infusion fluid 18 is introduced into volume 24 of eye 12. This warming method can be practiced with a deoxygenated infusion fluid 18 by replacing the silicon tubing with an oxygen impermeable tubing, thus keeping oxygen out of infusion fluid 18 during its transit from container 14 to eye 12. In another embodiment, infusion fluid 18 is warmed by a fluid warming device, such as described above.

In one embodiment, a method for delivering an infusion fluid from a container into a volume defined within an eye is provided. The method includes removing oxygen from within the infusion fluid as the infusion fluid flows from a chamber defined by the container into the volume through a first tube providing fluid communication between the chamber and the volume. At least a portion of the infusion fluid is infused into the volume through a first passage defined by the first tube. A second tube is positioned about the first tube. The second tube defines a second passage between an outer surface of the first tube and an inner surface of the second tube. At least one substantially oxygen-free inert gas is contained within the second tube to facilitate maintaining the infusion fluid in a low oxygen state. In a further embodiment, a stream of the at least one substantially oxygen-free inert gas is urged to move or flow through the second passage to facilitate deoxygenating the infusion fluid within the first passage. In a particular embodiment, the stream of the at least one substantially oxygen-free inert gas includes nitrogen gas, which flows through the second tube for facilitating diffusion of oxygen within the infusion fluid through the first tube and into the stream of nitrogen gas. In a further embodiment, the stream of the at least one substantially oxygen-free inert gas flows through the second tube in a direction substantially opposite a direction in which the infusion fluid flows through the first tube. The method facilitates removal of oxygen from the infusion fluid such that an oxygen level within the infusion fluid is reduced to about zero. A mixture of the initially substantially oxygen-free inert gas and oxygen is expelled to the atmosphere.

In a further embodiment, a method for infusing a deoxygenated fluid from a container into a volume defined within an eye includes infusing at least a portion of the deoxygenated fluid into the volume through a passage defined by a first tube providing fluid communication between a chamber defined by the container and the volume. A second tube is positioned about the first tube. The second tube defines a second passage between an outer surface of the first tube and an inner surface of the second tube. A vacuum is produced within the second tube using a suitable mechanism known to those skilled in the art and guided by the teachings herein provided to facilitate maintaining the deoxygenated fluid in a low oxygen state.

In yet a further embodiment, a method for delivering a deoxygenated fluid from a container into a volume defined within an eye includes infusing at least a portion of the deoxygenated fluid into the volume through a passage defined by a tube providing fluid communication between a chamber defined by the container and the volume. The tube is made of a substantially oxygen-impermeable material including, without limitation, a flexible oxygen-impermeable metal, plastic or polymer-based tubing material having a composition that serves as a barrier to oxygen.

The above-described apparatus and method for delivering infusion fluid, such as ophthalmic fluid, into an eye facilitates preventing oxygen from entering the infusion fluid flowing through the inner tube. More specifically, the apparatus and method facilitates removal of oxygen from the infusion fluid without opening the container and/or bubbling gas into the infusion fluid. As a result, the apparatus and method of the present invention facilitate the removal of oxygen from infusion fluid without undesirable contact with the infusion fluid, which may be a concern to regulatory authorities. Further, the above-described apparatus and method allow the use of currently available infusion fluids, while decreasing the oxygen level within the infusion fluid infused into the eye. In addition, supplements, such as reduced glutathione and/or ascorbic acid, can be added to the infusion fluid before surgery without concern of increasing the amount of oxygen delivered to the eye. Further, the apparatus may be configured to warm the infusion fluid to a suitable temperature, such as between about 30° C. and about 37° C. and, in a particular embodiment, to about 34° C.

Exemplary embodiments of an apparatus and method for delivering infusion fluid into an eye are described above in detail. The apparatus and method are not limited to the specific embodiments described herein, but rather, components of the apparatus and/or steps of the method may be utilized independently and separately from other components and/or steps described herein. Further, the described apparatus components and/or method steps can also be defined in, or used in combination with, other apparatus and/or methods, and are not limited to practice with only the apparatus and method as described herein.

While the invention has been described in terms of various specific embodiments, those skilled in the art will recognize that the invention can be practiced with modification within the spirit and scope of the claims.

What is claimed is:

1. An apparatus for delivering infusion fluid into an eye, said apparatus comprising:
a container defining a chamber configured to contain a quantity of infusion fluid;
a first tube defining a first passage providing fluid communication between said chamber and a volume defined within the eye, at least a portion of said quantity of infusion fluid infused into the volume through said first passage; and
a second tube positioned about said first tube and defining a second passage between an outer surface of said first tube and an inner surface of said second tube, a quantity of at least one substantially oxygen-free inert gas contained within said second tube to facilitate maintaining the infusion fluid in a low oxygen state.

2. An apparatus in accordance with claim 1 wherein a flow of said quantity of at least one substantially oxygen-free inert gas through said second tube facilitates diffusion of oxygen within said infusion fluid through said first tube and into said second passage.

3. An apparatus in accordance with claim 2 wherein said infusion fluid flows in a first direction through said first tube and said quantity of at least one substantially oxygen-free inert gas flows through said second tube in a second direction opposite said first direction.

4. An apparatus in accordance with claim 1 wherein said container comprises an oxygen impermeable storage bottle.

5. An apparatus in accordance with claim 1 wherein said infusion fluid comprises a deoxygenated ophthalmic fluid.

6. An apparatus in accordance with claim 1 wherein said infusion fluid comprises at least one supplement.

7. An apparatus in accordance with claim 6 wherein said at least one supplement comprises at least one of reduced glutathione and ascorbic acid.

8. An apparatus in accordance with claim 1 wherein said quantity of at least one substantially oxygen-free inert gas comprises one of nitrogen, a noble gas and combinations thereof.

9. A method for delivering an infusion fluid from a container into a volume defined within an eye, said method comprising removing oxygen from within the infusion fluid as the infusion fluid flows from a chamber defined by the container into the volume through a first tube providing fluid communication between the chamber and the volume.

10. A method in accordance with claim 9 wherein removing oxygen from within the infusion fluid further comprises:
infusing at least a portion of the infusion fluid into the volume through a first passage defined by the first tube; and
positioning a second tube about the first tube, the second tube defining a second passage between an outer surface of the first tube and an inner surface of the second tube, the second tube containing at least one substantially oxygen-free inert gas to facilitate maintaining the infusion fluid in a low oxygen state.

11. A method in accordance with claim 10 further comprising moving a stream of the at least one substantially oxygen-free inert gas through the second passage to facilitate deoxygenating the infusion fluid within the first passage.

12. A method in accordance with claim 11 wherein moving a stream of the at least one substantially oxygen-free inert gas through the second passage further comprises moving the stream comprising nitrogen gas through the second tube for facilitating diffusion of oxygen within the infusion fluid through the first tube and into the stream of nitrogen gas.

13. A method in accordance with claim 11 wherein moving a stream of at least one substantially oxygen-free inert gas through the second passage further comprises moving the stream through the second tube in a direction substantially opposite a direction in which the infusion fluid flows through the first tube.

14. A method in accordance with claim 10 further comprising limiting oxygen diffusion through the first tube into the infusion fluid.

15. A method in accordance with claim 9 further comprising reducing an oxygen level within the infusion fluid to less than about 10 mmHg.

16. A method in accordance with claim 9 further comprising expelling a mixture of the initially at least one substantially oxygen-free inert gas and oxygen to the atmosphere.

17. A method in accordance with claim 9 further comprising warming the infusion fluid to a temperature between about 30° C. and about 37° C.

18. A method for delivering ophthalmic fluid into an eye, said method comprising:
providing a container defining a chamber configured to contain deoxygenated ophthalmic fluid;
infusing a quantity of ophthalmic fluid from the container into the eye through a first tube defining a first passage providing fluid communication between the chamber and a volume defined within the eye; and
positioning a second tube about the first tube, the second tube defining a second passage between an outer surface of the first tube and an inner surface of the second tube, the second tube containing at least one substantially oxygen-free inert gas to facilitate maintaining the ophthalmic fluid in a low oxygen state.

19. A method in accordance with claim 18 further comprising moving a stream of the at least one substantially oxygen-free inert gas through the second passage to facilitate deoxygenating the ophthalmic fluid within the first passage.

20. A method in accordance with claim 19 wherein moving a stream of the at least one substantially oxygen-free inert gas through the second passage facilitates oxygen diffusion through the first tube into the stream of the at least one substantially oxygen-free inert gas.

21. A method in accordance with claim 19 wherein moving a stream of the at least one substantially oxygen-free inert gas through the second passage further comprises moving the stream consisting essentially of nitrogen gas through the second tube to facilitate diffusion of oxygen within the ophthalmic fluid through the first tube and into the stream of nitrogen gas.

22. A method in accordance with claim 19 wherein moving a stream of the at least one substantially oxygen-free inert gas through the second passage further comprises moving the stream through the second tube in direction substantially opposite a direction in which the ophthalmic fluid flows through the first tube.

23. A method in accordance with claim 19 further comprising reducing an oxygen level within the ophthalmic fluid to less than 10 mmHG.

24. A method in accordance with claim 18 further comprising warming the ophthalmic fluid to a temperature between about 30° C. and about 37° C.

25. A method for infusing a deoxygenated fluid from a container into a volume defined within an eye, said method comprising:
infusing at least a portion of the deoxygenated fluid into the volume through a passage defined by a first tube providing fluid communication between a chamber defined by the container and the volume; and
positioning a second tube about the first tube, the second tube defining a second passage between an outer surface of the first tube and an inner surface of the second tube, a vacuum produced within the second tube to facilitate maintaining the deoxygenated fluid in a low oxygen state.

26. A method for delivering a deoxygenated fluid from a container into a volume defined within an eye, said method comprising infusing at least a portion of the deoxygenated fluid into the volume through a passage defined by a tube providing fluid communication between a chamber defined by the container and the volume, the tube made of a substantially oxygen-impermeable material.

27. A method in accordance with claim 26 further comprising warming the deoxygenated fluid to a temperature between about 30° C. and about 37° C.
